(19) United States
(12) Patent Application Publication
(10) Pub. No.: US 2020/0365279 A1
(43) Pub. Date: Nov. 19, 2020

(54) SYSTEMS AND METHODS FOR SEARCHING AND PRESENTING MEDICAL RESULTS DERIVED FROM A CORPUS OF MEDICAL LITERATURE VIA ARTIFICIAL INTELLIGENCE

(71) Applicant: Evid Science, Inc., El Segundo, CA (US)

(72) Inventors: Matthew Michelson, La Cañada, CA (US); Michael Ross, Los Angeles, CA (US)

(21) Appl. No.: 16/412,291

(22) Filed: May 14, 2019

(51) Int. Cl. G16H 50/70; G06F 16/248; G06F 16/245; G16H 10/60; G06N 20/00

(52) U.S. Cl. CPC G16H 50/70; G06F 16/248; G06N 20/00; G16H 10/60; G06F 16/245

(57) ABSTRACT

Systems and methods for searching and presenting medical results derived from a corpus of medical literature via artificial intelligence are disclosed. Exemplary implementations may: obtain a set of medical results derived from a corpus of medical literature; present a user interface configured to facilitate searching and presenting the set of medical results; obtain a user-provided search query specifying one or more values of one or more attributes of the set of medical results; search the set of medical results for values of the attributes matching the user-provided search query; display a set of user-selectable filter elements through which the search results may be further refined; and/or perform other operations.

### FIELD OF THE DISCLOSURE

The present disclosure relates to systems and methods for searching and presenting medical results derived from a corpus of medical literature via artificial intelligence.

### BACKGROUND

It is important for doctors and other individuals in the health field to be up-to-date on the latest, effective therapies. Some techniques are known that generate a repository of medical results based on a corpus of medical literature, which may also be refreshed as new literature is published. The medical literature may include, for example, studies, research papers, randomized controlled trials, and/or other literature. Some techniques may utilize artificial intelligence systems to extract details from the literature and represent the extracted information in a structured form providing access to the medical results by both machines and people. By automatically extracting outcomes data, sometimes called results or evidence, from the medical literature, such a structured data set of results/evidence may be created at a scale covering a multitude of the published literature.

### SUMMARY

One aspect of the present disclosure relates to a system configured for searching and presenting medical results derived from a corpus of medical literature. One or more implementations address problems identified from prior systems which may generate a repository of medical results based on a corpus of medical literature using artificial intelligence or other machine learning. These problems may include an insufficient manner in which the medical results are made searchable and/or presentable to users. The prior techniques for presentation may be insufficient in that they may not go beyond mere regurgitation of the derived results which may not allow some users to use/view the results in a meaningful manner. For example, a simple spreadsheet of the results and/or web-based presentation table of results may be difficult to parse through, find relevant information, and/or may otherwise may not be user friendly. Further, the prior techniques may facilitate only a limited ability to search for specific results.

One or more implementations of a system for searching and presenting medical results derived from a corpus of medical literature may include one or more hardware processors configured by machine-readable instructions. The processor(s) may be configured to obtain a set of medical results derived from a corpus of medical literature. The set of medical results may be stored in a structured database. The structured database may refer a particular, well-defined format of storage. By way of non-limiting illustration, the medical results may be represented by values of attributes stored within the database. The attributes may include one or more of an intervention attribute, a group attribute, an outcome attribute, a problem attribute, a context attribute, and/or other attributes.

The processor(s) may be configured to present a user interface configured to facilitate searching and presenting of medical results from the set of medical results. The user interface may include one or more of an input portion, a display portion, a filter portion, and/or other portions. The input portion may be configured to obtain user-provided search queries. The display portion may be configured to display search results matching the user-provided search queries. The filter portion may be configured to obtain user entry and/or selection of user-selectable filter elements to refine the search results.

The processor(s) may be configured to obtain, via the input portion, one or more user-provided search queries. A user-provided search query may specify one or more of the values of one or more of the attributes. The processor(s) may be configured to search the set of medical results for values of the attributes matching and/or associated with the user-provided search query(s). The processor(s) may be configured to obtain, via the set of medical results, search results for the user-provided search query(s). The processor(s) may be configured to display, via the display portion, the search results. The search results may be displayed in a grid of cells and/or in other arrangement. The grid of cells may be arranged in attribute-named columns and rows. The rows may include cells specifying individual values in the set of values of corresponding attributes named in the columns. The processor(s) may be configured to display, via the filter portion, a set of user-selectable filter elements corresponding to the set of attributes through which the search results may be further refined.

As used herein, any association (or relation, or reflection, or indication, or correspondency) involving servers, processors, client computing platforms, and/or another entity or object that interacts with any part of the system and/or plays a part in the operation of the system, may be a one-to-one association, a one-to-many association, a many-to-one association, and/or a many-to-many association or N-to-M association (note that N and M may be different numbers greater than 1).

As used herein, the term “obtain” (and derivatives thereof) may include active and/or passive retrieval, determination, derivation, transfer, upload, download, submission, and/or exchange of information, and/or any combination thereof. As used herein, the term “effectuate” (and derivatives thereof) may include active and/or passive causation of any effect. As used herein, the term “determine” (and derivatives thereof) may include measure, calculate, compute, estimate, approximate, generate, and/or otherwise derive, and/or any combination thereof.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 illustrates a system configured for searching and presenting medical results derived from a corpus of medical literature, in accordance with one or more implementations.

FIG. 2 illustrates a method for searching and presenting medical results derived from a corpus of medical literature, in accordance with one or more implementation

FIG. 3 illustrates an exemplary user interface, in accordance with one or more implementations.

FIG. 4 illustrates an exemplary user interface, in accordance with one or more implementations.

FIG. 5 illustrates an exemplary display of search results as an electronic document, in accordance with one or more implementations.

### DETAILED DESCRIPTION

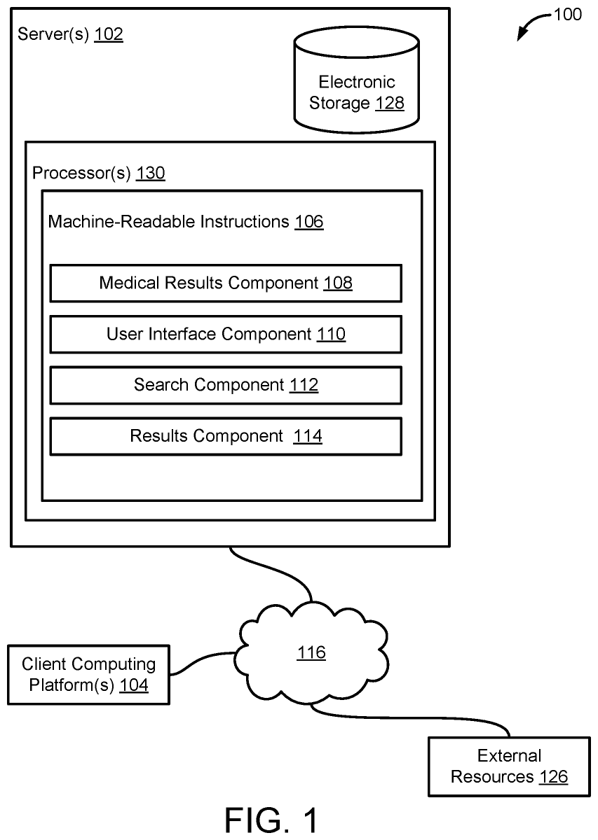

FIG. 1 illustrates a system 100 configured for searching and presenting medical results, in accordance with one or more implementations. One or more implementations address problems identified from prior systems which may generate a repository of medical results based on a corpus of medical literature using artificial intelligence (Al) or other machine learning. By way of non-limiting illustration, the results extracted from the Al may be formatted in a far more readable format. Rather than simply presenting rows in a database, the results may be formatted based on user input to be readable, usable, and include more relevant information (based on user search queries). Users may refine the results by searching and filtering to what they care about most.

In some implementations, system 100 may include one or more of one or more servers 102, one or more client computing platforms 104, external resources 126, and/or other components. Server(s) 102 may be configured to communicate with one or more client computing platforms 104 according to a client/server architecture and/or other architectures. Client computing platform(s) 104 may be configured to communicate with other client computing platforms via server(s) 102 and/or according to a peer-to-peer architecture and/or other architectures. Users may access system 100 via client computing platform(s) 104.

Server(s) 102 may include one or more of non-transitory electronic storage 128, one or more processors 130 configured by machine-readable instructions 106, and/or other components. Machine-readable instructions 106 may include one or more instruction components. The instruction components may include computer program components. Executing the machine-readable instructions 106 may cause server(s) 102 to facilitate searching and presenting medical results derived from a corpus of medical literature. The instruction components may include one or more of a medical results component 108, a user interface component 110, a search component 112, a results component 114, and/or other instruction components.

The medical results component 108 may be configured to obtain a set of medical results derived from a corpus of medical literature. The medical results may have been derived from a corpus of medical literature via artificial intelligence and/or other techniques. The set of medical results may be stored in a structured database The set of medical results be structured by virtue of the medical results being represented by values of pre-defined attributes. The attributes may include one or more of an intervention attribute, a group attribute, an outcome attribute, a problem attribute, a context attribute, and/or other attributes.

A value of an intervention attribute may specify one or more of a drug used, an amount of a drug administered, a time period over which a drug was administered, a procedure performed, and/or other information.

A value of a group attribute may specify patient-level information and/or other information. The patient-level information may include one or more of age, age range, demographic, inclusion and/or exclusion study criteria, sex, and/or other information.

A value of an outcome attribute may specify one or more of a result of treatment, a time period in which a certain result was obtained, and/or other information. By way of non-limiting illustration, a result may include one or more of an adverse event, remission, hospitalization, and/or other information.

A value of a problem attribute may specify one or more of a disease present, disease study population criteria, and/or other information.

A value of a context attribute may specify information derived either directly and/or indirectly from the medical literature from which the medical results were derived. Directly derived may refer to information present on the face of the medical literature. Indirectly derived may refer to information that may be systematically and/or logically determined from the information present on the face of the medical literature. By way of non-limiting illustration, a value of a context attribute may include one or more of a citation to medical literature, a link to the literature, relative risk, a classification of the medical literature (e.g., random control study, clinical trial, etc.), and/or other information.

It is noted that the above descriptions of various attributes having values defining medical results within a database are for illustrative purposes only and not to be considered limiting. Instead, those skilled in the art may recognize other values of these attributes and/or other attributes which may be used to define the stored data in the context of creating usable and meaning representations of medical results.

User interface component 110 may be configured to effectuate presentation of individual user interfaces on individual client computing platforms of one or more client computing platforms 104. For example, the individual client computing platforms may access a user interface over network 116. A given user interface may be configured to facilitate searching and presenting medical results from the set of medical results (see medical results component 108). The user interface may include one or more user interface portions. By way of non-limiting illustration, a user interface may include one or more of an input portion, a display portion, a filter portion, and/or other portions. Individual portions may include one or more user interface elements configured to facilitate user interaction with the user interface. By way of non-limiting illustration, user interface elements may include one or more of text input fields, drop down menus, check boxes, display windows, virtual buttons, and/or other elements configured to facilitate user interaction.

An input portion of a user interface may be configured to obtain user-provided search queries. A user-provided search query may specify one or more of the values of one or more of the attributes. The input portion may comprise a text input field configured to accept user entry and/or selection of text (e.g., via a keyboard, speech to text, or the like). By way of non-limiting illustration, a user-provided search query may include a text string conveying one or more values of one or more attributes of medical results.

A display portion may be configured to display search results matching the user-provided search queries. The display portion may comprise a display window configured to present the search results visually in the form of text, hyperlinks, images, generated graphs, and/or other ways of visual presentation. The search results may be arranged in a grid of cells and/or by other display arraignment. The grid of cells may be arranged by columns and rows. The columns may be attribute-named columns individually representing an attribute. The rows may include cells specifying individual values of corresponding attributes named in the columns. In some implementations, individual rows have values that individually correspond to one or more of a specific piece of literature (or group of literature), a specific literature classification type, a specific study, one or more derived attributes, a specific attribute, and/or other information.

A filter portion may be configured to obtain user entry and/or selection of user-selectable filter elements to refine the search results. The individual user-selectable filter elements may specify individual values of individual attributes. The selection of the individual user-selectable filter elements may refine the search results based on the individual values of individual attributes specified by the individual user-selectable filter elements. The selection of the individual user-selectable filter elements may refine the search results by narrowing the search results to the user-provided search query in addition to the individual values of individual attributes specified by the selected user-selectable filter elements. In some implementations, the user-selectable filter elements may be represented by check-boxes and/or other user interface elements (e.g., drop-down menu and/or other user interface elements).

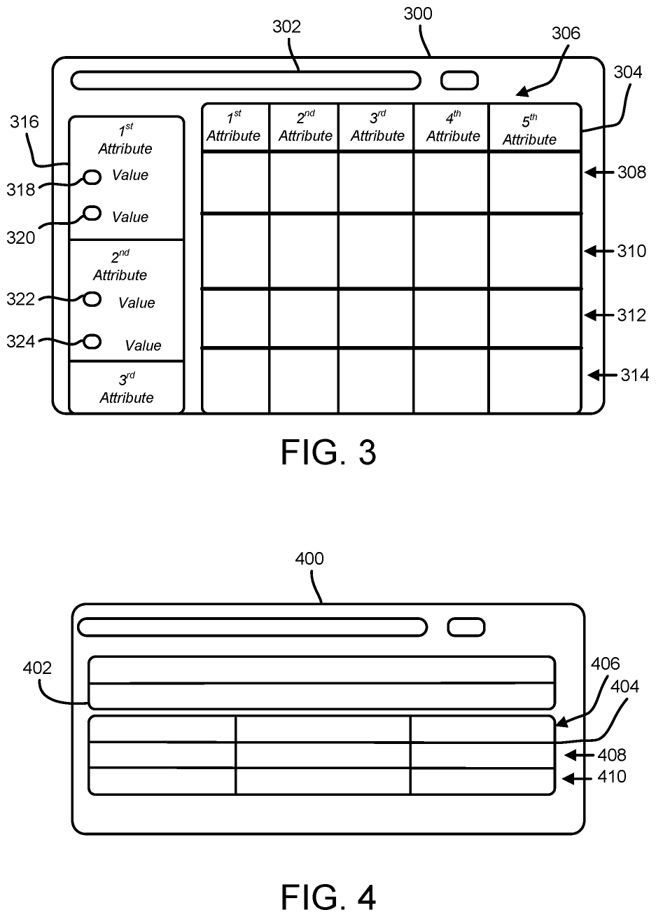

FIG. 3 illustrates an exemplary user interface 300, in accordance with one or more implementations. The user interface 300 may include one or more user interface portions. By way of non-limiting illustration, user interface 300 may include one or more of an input portion 302, a display portion 304, a filter portion 316, and/or other portions.

The input portion 302 may comprise a text input field and/or other user interface element. The display portion 304 may comprise a display window configured in a grid comprising attribute-named columns 306 and a set of rows 308-314. The rows may include cells specifying individual values of corresponding attributes named in the columns 306. In some implementations, the grid formed by the columns and rows may include demarking lines that form specific cells, as shown, and/or there may not be any demarking lines between cells, yet the visually arrangement may still convey to the user the relationship between cells of the rows and the columns.

The filter portion 316 may include a set of user-selectable filter elements 318-324 which may be associated with a set of values (labeled generically as “value” in the figure for illustrative purposes) of individual attributes included in the attributed-named columns. Here, the set of user-selectable filter elements 318-324 are shown as individual check-boxes, however, other user interface elements may be utilized.

It is noted that the size of user interface 300, disposition of the various portions, and/or other features of the user interface 300 are shown for illustrative purposes only and are not to be considered limiting. For example, other arrangements of the various portions or components of user interfaces described herein may be utilized which are still with the scope of this disclosure (see, e.g., FIG. 4 and accompanying description herein). Further, the user interface 300 may include more content then shown and may be scrollable by a user to access results that may not appear in the displayable area of the user interface 300.

Returning to FIG. 1, search component 112 may be configured to obtain, via the input portion, one or more user-provided search queries. Search component 112 may be configured to search the set of medical results obtained by medical results component 108 for values of the attributes matching individual ones of the user-provided search queries. A match may refer to one or more of an exact match, a logically similar match, and/or other consideration of determining search results. A logically similar match may refer to a match that may not be exact (e.g., have the exact same text or phrasing), but may include text or phrases represented by different words otherwise mean the same thing. By way of non-limiting illustration, a logically similar match may be found between a query of “Crohn's disease” and a medical result specifying “crohn disease.” By way of non-limiting illustration, a logically similar match may be found between a query of “no signs or symptoms of disease” and a medical result specifying “remission.”

Results component 114 may be configured to obtain, via the set of medical results, the search results for individual ones of the user-provided search queries. The search results for an individual user-provided search query may include a set of values of a set of attributes from the set of medical results matching the individual user-provided search query.

User interface component 110 may be configured to display, within a display portion of a user interface, the search results of individual user-provided search queries. The search results may be displayed in the grid of cells and/or by other display technique. The grid of cells may be arranged in attribute-named columns and rows of cells specifying individual values in the set of values of corresponding attributes named in the columns.

User interface component 110 may be configured to display, via the filter portion, a set of user-selectable filter elements corresponding to the set of attributes (included in the search results) through which the search results may be be further refined. The individual user-selectable filter elements may specify individual values of individual attributes in the set of attributes of the search results. The selection of the individual user-selectable filter elements may refine the search results based on the individual values of individual attributes specified by the individual user-selectable filter elements. The refining of the search results based on the individual values of individual attributes specified by the individual user-selectable filter elements may reduce the search results to include a subset of the set of values of the set of attributes of the search query. The subset of the set of values may include values matching both the user-provided search query and the individual values of individual attributes specified by the selected user-selectable filter elements.

In some implementations, user interface component 110 may be configured to modify individual values of the attributes returned by the search component 112. The modification may be performed prior to displaying search results, in response to user request, and/or other considerations. The modifying the individual values may include changing the representation of the individual values in a display portion of a user interface. Changing the representation may include converting the value into a canonical representation. By way of non-limiting illustration, using the above example of a query of “Crohn's disease” and a medical result specifying “crohn disease,” the displayed search results may modify the medical result to recite “Crohn's disease” (e.g., “Crohn's disease” being the canonical representation of other representations of the term). By way of non-limiting illustration, using the above example of a query of “no signs or symptoms of disease” and a medical result specifying “remission,” the displayed search results may modify displayed medical results to recite “remission” (e.g., “remission” being the canonical representation of other representations of the term). By way of non-limiting illustration, phrases describing percentages or fractions may be changed to be displayed in a numerical form (e.g., the numerical form being the canonical representation, or vice versa). For example, the phrase “12 out of 16” may be changed to 12/16 (or ¾%, 75%, and/or other representation)

User interface component 110 may be configured to receive user input to display comparisons of sets of values of attributes to other sets of values of attributes. By way of non-limiting illustration, user input may be provided to compare a specific therapy to a placebo treatment (e.g., comparing different values for an interventions attribute) against a particular disease, a particular outcome, and/or other information. The search component 112 may be configured to may obtain comparison results including values of attributes which include the specific therapy and placebo as values of intervention attributes and find other values of other attributes associated with the specific therapy and placebo. Other types of comparisons are contemplated.

In some implementations, user interface component 110 may be configured facilitate generating electronic documents from search results. An electronic document may be in the form of a spreadsheet representation of the search results and/or other representation. The electronic document may provide a technique to share (e.g., over network 116) and/or print results as desired. In some implementations, the user interface component 110 may concatenate multiple attributes into a common attributed-named column. A cell under the column may include a concatenated representation of multiple values corresponding to the multiple attributes. By way of non-limiting illustration, the displayed results might indicate the “numerator attribute” has a value of “12,” the “denominator attribute” has a value of “16,” and the cell under a new column called “result” may be concatenated to recite “12 of 16.”

FIG. 4 illustrates another implementation of a user interface 400, according to one or more implementations described herein. The user interface 400 may include a first display portion 402, a second display portion 404, and/or other portions and/or components. Here, the second display portion 404 may comprise a display window configured in a grid comprising attribute-named columns 406 and a set of rows 408 and 410. The rows may include cells specifying individual values of corresponding attributes named in the columns 406. The first display 404 may include a display window configured to display other values of other attributes, concatenated versions of values of attributes, and/or other information relevant to the values displayed in the second display portion 404. By way of non-limiting illustration, a row of the first display portion 402 may present a one to two sentence summary of the results shown in the second display portion 404, while another row may present a title, link, or summary of the literature through which results were derived.

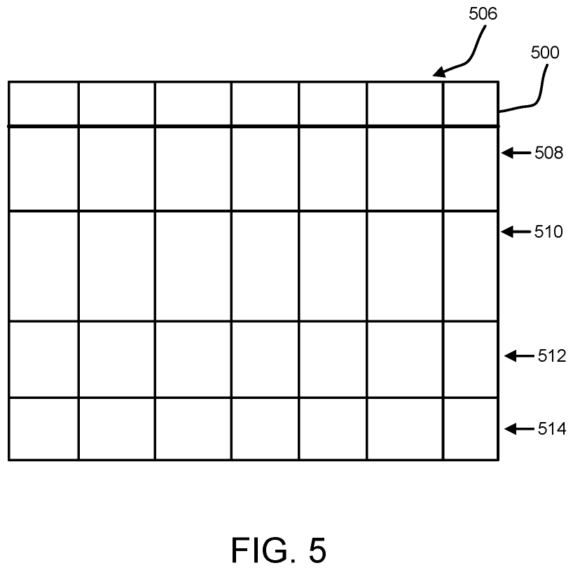

FIG. 5 illustrates an exemplary display of search results as an electronic document 500, in accordance with one or more implementations. The document 500 may comprise a spreadsheet representation of search results comprising a grid. The grid may include attribute-named columns 506 and a set of rows 508-514. The rows may include cells specifying individual values of corresponding attributes named in the columns 506.

Returning to FIG. 1, in some implementations, server(s) 102, client computing platform(s) 104, and/or external resources 126 may be operatively linked via one or more electronic communication links. For example, such electronic communication links may be established, at least in part, via a network 116 such as the Internet and/or other networks. It will be appreciated that this is not intended to be limiting, and that the scope of this disclosure includes implementations in which server(s) 102, client computing platform(s) 104, and/or external resources 126 may be operatively linked via some other communication media.

A given client computing platform 104 may include one or more processors configured to execute computer program components. The computer program components may be configured to enable an expert or user associated with the given client computing platform 104 to interface with system 100 and/or external resources 126, and/or provide other functionality attributed herein to client computing platform(s) 104. By way of non-limiting example, the given client computing platform 104 may include one or more of a desktop computer, a laptop computer, a handheld computer, a tablet computing platform, a NetBook, a Smartphone, a gaming console, and/or other computing platforms.

External resources 126 may include sources of information outside of system 100, external entities participating with system 100, and/or other resources. In some implementations, some or all of the functionality attributed herein to external resources 126 may be provided by resources included in system 100.

Server(s) 102 may include electronic storage 128, one or more processors 130, and/or other components. Server(s) 102 may include communication lines, or ports to enable the exchange of information with a network 116 and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 128 may comprise non-transitory storage media that electronically stores information. The electronic storage media of electronic storage 128 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 128 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 128 may include one or more virtual storage resources (e.g., cloud storage, a virtual private network, and/or other virtual storage resources). Electronic storage 128 may store software algorithms, information determined by processor(s) 130, information received from server(s) 102, information received from client computing platform(s) 104, and/or other information that enables server(s) 102 to function as described herein.

Processor(s) 130 may be configured to provide information processing capabilities in server(s) 102. As such, processor(s) 130 may include one or more of a digital processor, an analog processor, a digital circuit designed to process information, an analog circuit designed to process information, a state machine, and/or other mechanisms for electronically processing information. Although processor(s) 130 is shown in FIG. 1 as a single entity, this is for illustrative purposes only. In some implementations, processor(s) 130 may include a plurality of processing units. These processing units may be physically located within the same device, or processor(s) 130 may represent processing functionality of a plurality of devices operating in coordination. Processor(s) 130 may be configured to execute components 108, 110, 112, and/or 114, and/or other components. Processor(s) 130 may be configured to execute components 108, 110, 112, and/or 114, and/or other components by software; hardware; firmware; some combination of software, hardware, and/or firmware; and/or other mechanisms for configuring processing capabilities on processor(s) 130. As used herein, the term “component” may refer to any component or set of components that perform the functionality attributed to the component. This may include one or more physical processors during execution of processor readable instructions, the processor readable instructions, circuitry, hardware, storage media, or any other components.

It should be appreciated that although components 108, 110, 112, and/or 114 are illustrated in FIG. 1 as being implemented within a single processing unit, in implementations in which processor(s) 130 includes multiple processing units, one or more of components 108, 110, 112, and/or 114 may be implemented remotely from the other components. The description of the functionality provided by the different components 108, 110, 112, and/or 114 described below is for illustrative purposes, and is not intended to be limiting, as any of components 108, 110, 112, and/or 114 may provide more or less functionality than is described. For example, one or more of components 108, 110, 112, and/or 114 may be eliminated, and some or all of its functionality may be provided by other ones of components 108, 110, 112, and/or 114. As another example, processor(s) 130 may be configured to execute one or more additional components that may perform some or all of the functionality attributed below to one of components 108, 110, 112, and/or 114.

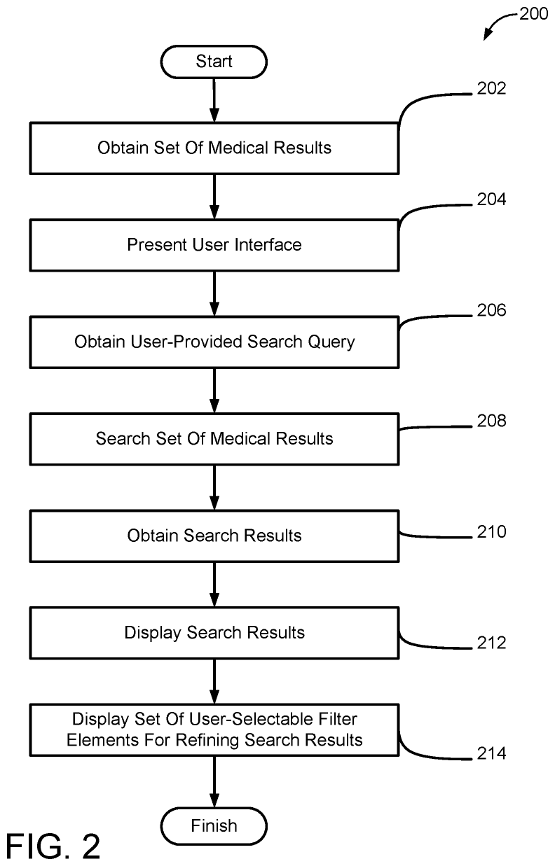

FIG. 2 illustrates a method 200 for searching and presenting medical results derived from a corpus of medical literature via artificial intelligence, in accordance with one or more implementations. The operations of method 200 presented below are intended to be illustrative. In some implementations, method 200 may be accomplished with one or more additional operations not described, and/or without one or more of the operations discussed. Additionally, the order in which the operations of method 200 are illustrated in FIG. 2 and described below is not intended to be limiting.

In some implementations, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

An operation 202 may include obtaining a set of medical results derived from a corpus of medical literature. The set of medical results may be represented by values of attributes. The set of medical results may be stored in a structured database. The attributes may include one or more of an intervention attribute, a group attribute, an outcome attribute, a problem attribute, a context attribute, and/or other attributes. Operation 202 may be performed by one or more hardware processors configured by machine-readable instructions including a component that is the same as or similar to medical results component 108, in accordance with one or more implementations.

An operation 204 may include presenting a user interface configured to facilitate searching and presenting the set of medical results. The user interface may include one or more of an input portion, a display portion, a filter portion, and/or other portions. The input portion may be configured to obtain user-provided search queries. The display portion may be configured to display search results matching the user-provided search queries. The filter portion may be configured to obtain user entry and/or selection of user-selectable filter elements to refine the search results. Operation 204 may be performed by one or more hardware processors configured by machine-readable instructions including a component that is the same as or similar to user interface component 110, in accordance with one or more implementations.

An operation 206 may include obtaining, via the input portion, a user-provided search query specifying one or more of the values of one or more of the attributes. Operation 206 may be performed by one or more hardware processors configured by machine-readable instructions including a component that is the same as or similar to user interface component 110, in accordance with one or more implementations.

An operation 208 may include searching the set of medical results for values of the attributes matching the user-provided search query. Operation 208 may be performed by one or more hardware processors configured by machine-readable instructions including a component that is the same as or similar to search component 112, in accordance with one or more implementations.

An operation 210 may include obtaining, via the set of medical results, the search results for the user-provided search query. The search results may include a set of values of a set of attributes from the set of medical results matching the user-provided search query. Operation 210 may be performed by one or more hardware processors configured by machine-readable instructions including a component that is the same as or similar to results component 114, in accordance with one or more implementations.

An operation 212 may include displaying, via the display portion, the search results. The search results may be displayed in a grid of cells arranged in attribute-named columns and rows of cells specifying individual values in the set of values of corresponding attributes named in the columns. Operation 212 may be performed by one or more hardware processors configured by machine-readable instructions including a component that is the same as or similar to user interface component 110, in accordance with one or more implementations.

An operation 214 may include displaying, via the filter portion, a set of user-selectable filter elements corresponding to the set of attributes through which the search results may be further refined. Individual user-selectable filter elements may specify individual values of individual attributes in the set of attributes. Selection of the individual user-selectable filter elements may refine the search results based on the individual values of individual attributes specified by the individual user-selectable filter elements. The refining of the search results based on the individual values of individual attributes specified by the individual user-selectable filter elements may reduce the search results to include a subset of the set of values of the set of attributes. The subset of the set of values may include values matching one or both of the user-provided search query or the individual values of individual attributes specified by the individual user-selectable filter elements. Operation 214 may be performed by one or more hardware processors configured by machine-readable instructions including a component that is the same as or similar to user interface component 110, in accordance with one or more implementations.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed is:

1. A system configured to search and present medical results derived from a corpus of medical literature, the system comprising:
one or more physical processors configured by machine-readable instructions to:
obtain a set of medical results derived from a corpus of medical literature, the set of medical results being represented by values of attributes;
effectuate presentation of a user interface configured to facilitate searching and presenting the set of medical results, the user interface including an input portion, a display portion, and a filter portion, the input portion being configured to obtain user-provided search queries, the display portion being configured to display search results matching the user-provided search queries, the filter portion being configured to obtain user entry and/or selection of user-selectable filter elements to refine the search results;
obtain, via the input portion, a user-provided search query specifying one or more of the values of one or more of the attributes;
search the set of medical results for values of the attributes matching the user-provided search query;
obtain, via the set of medical results, the search results for the user-provided search query, the search results including a set of values of a set of attributes from the set of medical results matching the user-provided search query;
effectuate presentation, via the display portion, of the search results, the search results being displayed in a grid of cells arranged in attribute-named columns and rows of cells specifying individual values in the set of values of corresponding attributes named in the columns; and
effectuate presentation, via the filter portion, of a set of user-selectable filter elements corresponding to the set of attributes through which the search results will be further refined, individual user-selectable filter elements specifying individual values of individual attributes in the set of attributes, wherein selection of the individual user-selectable filter elements refine the search results based on the individual values of individual attributes specified by the selected individual user-selectable filter elements.

2. The system of claim 1, wherein refining the search results based on the individual values of individual attributes specified by the individual user-selectable filter elements reduces the search results to include a subset of the set of values of the set of attributes, wherein the subset of the set of values includes values matching both the user-provided search query and the individual values of individual attributes specified by the selected individual user-selectable filter elements.

3. The system of claim 1, wherein the attributes include one or more of an intervention attribute, a group attribute, an outcome attribute, a problem attribute, or a context attribute.

4. The system of claim 3, wherein a value of a context attribute specifies information derived directly and/or indirectly from the medical literature.

5. The system of claim 4, wherein information derived directly and/or indirectly from the medical literature includes one or more of a citation, a hyperlink to literature, relative risk, inclusion or exclusion criteria, or literature classification.

6. The system of claim 1, wherein the user interface is accessible to client computing platforms associated with users over a network.

7. The system of claim 1, wherein the one or more physical processors are further configured by the machine-readable instructions to modify individual values of the attributes.

8. The system of claim 7, wherein modifying includes changing how the values are represented.

9. The system of claim 1, wherein the one or more physical processors are further configured by the machine-readable instructions to receive user input to display comparisons of sets of values of attributes to other sets of values of the attributes.

10. The system of claim 1, wherein the one or more physical processors are further configured by the machine-readable instructions to concatenate multiple values into an individual representation of the multiple values.

11. A method for searching and presenting medical results derived from a corpus of medical literature via artificial intelligence, the method comprising:
obtaining a set of medical results derived from a corpus of medical literature, the set of medical results being represented by values of attributes;
presenting a user interface configured to facilitate searching and presenting the set of medical results, the user interface including an input portion, a display portion, and a filter portion, the input portion being configured to obtain user-provided search queries, the display portion being configured to display search results matching the user-provided search queries, the filter portion being configured to obtain user entry and/or selection of user-selectable filter elements to refine the search results;
obtaining, via the input portion, a user-provided search query specifying one or more of the values of one or more of the attributes;
searching the set of medical results for values of the attributes matching the user-provided search query;
obtaining, via the set of medical results, the search results for the user-provided search query, the search results including a set of values of a set of attributes from the set of medical results matching the user-provided search query;
displaying, via the display portion, the search results, the search results being displayed in a grid of cells arranged in attribute-named columns and rows of cells specifying individual values in the set of values of corresponding attributes named in the columns; and
displaying, via the filter portion, a set of user-selectable filter elements corresponding to the set of attributes through which the search results will be further refined, individual user-selectable filter elements specifying individual values of individual attributes in the set of attributes, wherein selection of the individual user-selectable filter elements refine the search results based on the individual values of individual attributes specified by the selected individual user-selectable filter elements.

12. The method of claim 11, wherein refining the search results based on the individual values of individual attributes specified by the individual user-selectable filter elements reduces the search results to include a subset of the set of values of the set of attributes, wherein the subset of the set of values includes values matching both the user-provided search query and the individual values of individual attributes specified by the selected individual user-selectable filter elements.

13. The method of claim 11, wherein the attributes include one or more of an intervention attribute, a group attribute, an outcome attribute, a problem attribute, or a context attribute.

14. The method of claim 13, wherein a value of a context attribute specifies information derived directly and/or indirectly from the medical literature.

15. The method of claim 14, wherein information derived directly and/or indirectly from the medical literature includes one or more of a citation, a hyperlink to literature, relative risk, inclusion or exclusion criteria, or literature classification.

16. The method of claim 11, wherein the user interface is accessible to client computing platform associated with users over a network.

17. The method of claim 11, further comprising modifying individual values of the attributes.

18. The method of claim 17, wherein modifying includes changing how the values are represented.

19. The method of claim 11, further comprising receiving user input to display comparisons of sets of values of attributes to other sets of values of the attributes.

20. The method of claim 11, further comprising concatenating multiple values into an individual representation of the multiple values.
